(19) United States
(12) Patent Application Publication
(10) Pub. No.: US 2018/0230466 A1
(43) Pub. Date: Aug. 16, 2018

(54) METHODS FOR TREATING TUMORS

(71) Applicants: Centenary Institute of Cancer Medicine and Cell Biology, Camperdown (AU); Mirrx Therapeutics A/S, Vejle (DK); University of Sydney, Sydney (AU)

(72) Inventors: Jennifer Gamble, Double Bay (AU); Thorlief Møller, Vejle (DK); Mathew Vadas, Double Bay (AU)

(21) Appl. No.: 15/750,092

(22) PCT Filed: Aug. 5, 2016
(86) PCT No.: PCT/AU2016/050712

§ 371 Date: Feb. 2, 2018

(30) Foreign Application Priority Data
Aug. 5, 2015 (AU) 2015903130

(51) Int. Cl. C12N 15/113; A61K 31/7105; A61K 45/06; A61P 35/00

(52) U.S. Cl. CPC C12N 15/113; A61K 31/7105; A61K 45/06; A61P 35/00; C12N 2320/31; C12N 2310/3231; C12N 2310/321; C12N 2310/323; C12N 2310/113

(57) ABSTRACT

The present invention provides methods for increasing the sensitivity of a tumour to immunotherapy, chemotherapy or radiotherapy by administering an effective amount of an oligonucleotide that inhibits the binding of miR-27a, or a variant thereof, to its target mRNA. Oligonucleotides used in the invention are typically in the form of a blockmirs used as an adjunctive therapy to inhibit tumour growth, normalise and/or improve function of tumour vasculature, and/or promote immune cell infiltration of tumours.

### FIELD OF THE INVENTION

The present invention relates generally to the use of oligonucleotides as an adjunctive therapy in inhibition of tumour growth, for normalisation and/or improving function of tumour vasculature, and/or for promoting immune cell infiltration of tumours.

### BACKGROUND OF THE INVENTION

As a tumour grows its requirements for nutrients become greater and angiogenesis is induced to supply new vasculature to the tumour. This tumour vasculature differs from normal vasculature, being poorly organised and having aberrant vessel walls. Fewer tight junctions, gaps between endothelial cells, loosely attached pericytes, and an abnormal basement membrane and extracellular matrix result in a hyperpermeable endothelial barrier. This poor vascularisation contributes to a hypoxic microenvironment in the tumour which induces biochemical pathways that promote further tumour development, eg via vascular endothelial growth factor (VEGF) and hypoxia inducible factor (HIF). As such, hypoxia promotes angiogenesis, inflammation, cancer stem cell morphology, immunosuppression and anaerobic metabolism. The hypoxic environment can also induce metastasis of the tumour.

A further consequence of poor perfusion of the tumour is an impairment of therapeutic treatments, such as chemotherapy and immunotherapy, which rely on the vasculature for delivery. Anti-angiogenic agents have been used with some success to temporarily normalize tumour vasculature and alleviate hypoxia (see, for example, Jain, 2001 Nat. Med. 9, 685-693) and adjunctive therapy combining anti-angiogenic agents with chemotherapeutic agents has been shown to increase survival in some cancer patients. However anti-angiogenic agents can cause extensive damage to, including destruction of, tumour vessels and vessel normalisation does not persist. Thus current tumour treatments pose difficulties in their effective delivery, and high doses of therapeutic agents required in treatment regimens which may produce unwanted side effects.

Accordingly, there is a need for novel treatments that effectively target the tumour vasculature without causing the damaging effects of known anti-angiogenic therapies.

### SUMMARY OF THE INVENTION

Disclosed herein is the use of oligonucleotides for modulating tumour stroma, tumour vasculature, metastasis and sensitivity to treatment, wherein the oligonucleotides comprise sequences complementary to and capable of binding to, the sequence shown in SEQ ID NO: 1.

In a first aspect the present invention provides a method for increasing the sensitivity of a tumour to immunotherapy, chemotherapy or radiotherapy, wherein the method comprises administering to a subject in need thereof an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof, or a miRNA comprising a seed region comprising the sequence UCACAG, to said RNA.

The method may further comprise immunotherapy, chemotherapy or radiotherapy of said tumour in said subject. In an embodiment, the oligonucleotide is administered to the subject prior to, concomitantly with, after, or otherwise in combination with, immunotherapy, chemotherapy or radiotherapy of said tumour.

In a particular embodiment, the oligonucleotide comprises a contiguous sequence complementary to at least 8 contiguous bases of an RNA sequence comprising SEQ ID NO: 2, or SEQ ID NO: 2 comprising 1, 2 or 3 substitutions, wherein the oligonucleotide inhibits the binding of miR-27a, a variant thereof or a miRNA comprising a seed region comprising the sequence UCACAG, to said RNA.

Typically the miR-27a miRNA is hsa-miR-27a comprising the nucleotide sequence set forth in SEQ ID NO: 9.

In an embodiment, the oligonucleotide comprises a contiguous sequence complementary to a sequence of at least or about 7 bases, at least or about 8 bases, at least or about 9 bases, at least or about 10 bases, at least or about 11 bases, at least or about 12 bases, at least or about 13 bases, at least or about 14 bases, at least or about 15 bases, at least or about 16 bases, at least or about 17 bases, at least or about 18 bases, at least or about 19 bases, at least or about 20 bases, at least or about 22 bases, at least or about 25 bases, at least or about 30 bases, or at least or about 35 bases of SEQ ID NO: 2, or SEQ ID NO: 2 comprising 1, 2 or 3 substitutions.

Typically the oligonucleotide binds to positions 22-27 of SEQ ID NO: 2.

In a particular embodiment, base pairing between the oligonucleotide and SEQ ID NO: 2 includes positions 8-28, 8-27, 9-27, 10-27, 11-27, 12-27, 13-27, 14-27, 15-27, 16-27, 17-27, 18-27, 19-27, 20-27, 21-27, 9-28, 10-28, 11-28, 12-28, 13-28, 14-28, 15-28, 16-28, 17-28, 18-28, 19-28, 20-28 or 21-28 of SEQ ID NO: 2.

In a further particular embodiment, the oligonucleotide comprises the sequence set forth in SEQ ID NO: 3 or SEQ ID NO: 4.

In a further particular embodiment, the oligonucleotide comprises one or more modified nucleobases. In exemplary embodiments, the modified nucleobase may be selected from an LNA nucleobase, a UNA nucleobase and a 2′ O-methyl nucleobase.

In a further particular embodiment, the oligonucleotide comprises a sequence set forth in SEQ ID NO: 5.

In a further particular embodiment, the immunotherapy comprises immune stimulation, comprising adoptive cell transfer or the administration of one or more anti-tumour or immune checkpoint antibodies, anti-tumour vaccines or other immune cell modulating agents. In an embodiment, adoptive cell transfer comprises the transfer of autologous tumour infiltrating lymphocytes. In a further embodiment, the anti-tumour antibodies comprise anti-PD-1 antibodies.

In a second aspect the present invention provides a method for modulating tumour metastasis, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

Typically, modulating tumour metastasis comprises reducing tumour metastasis.

In a third aspect the present invention provides a method for normalising tumour vasculature and/or improving vascular function in a tumour, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

In a particular embodiment, normalising the tumour vasculature and/or improving vessel function comprises or is characterized by one or more of: change in morphology of endothelial cells, change in VE-cadherin expression, selective loss of large vessels; increase in number of small vessels, increased pericyte coverage of vessels, altered collagen IV coverage of vessels, reduced vessel permeability, reduced vessel hypoxia, increased vessel perfusion, and enhanced infiltration of immune cells. In a particular embodiment the immune cells are lymphocytes. In a further embodiment the lymphocytes comprise CD8+ T cells, CD4+ T cells and/or NK cells.

In a fourth aspect the present invention provides a method for reducing tumour hypoxia, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

In a fifth aspect the present invention provides a method for increasing cell death of tumour cells, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

In a sixth aspect the present invention provides use of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA for the manufacture of a medicament for sensitising tumours, modulating tumour metastasis, normalising tumour vasculature and/or promoting cell death of tumour cells.

In particular embodiments of the above aspects, the tumour is a solid tumour.

### BRIEF DESCRIPTION OF THE DRAWINGS

Embodiments of the invention are described herein, by way of non-limiting example only, with reference to the following figures.

FIG. 1. Blockmir CD5-2 facilitates the infiltration of adoptively transferred CD8+ T cells into RIP-TAG tumours. (A) Representative composite images of tumours. DAPI (blue, nuclei) and CD8 (red, cytotoxic T cells) given either control Blockmir or Blockmir CD5-2 (2 different images are shown). (B) Quantification of the ratio of infiltrated CD8 T cells to cells in the visual field. Data represents mean±SEM. n=3 mice per group, paired t-test. (C) Quantification of the ratio of infiltrated CD8 T cells to cells in the visual field when removing one mouse of 3 that received the control Blockmir, since it showed an unusually high infiltration (>1 SD away from the mean). Data represents mean±SEM. n=2 mice treated with control and 3 mice treated with Blockmir CD5-2.

FIG. 2. Effect of Blockmir CD5-2 on the infiltration of endogenous CD8+ cytotoxic T cell into the B16F10 melanoma tumours. (A) Quantification of the number of CD8+ cells per field (two fields per mouse). Data represents mean±SEM. n=5 mice per group, paired t-test. (B) Representative composite images of B16F10 tumours. DAPI (blue, nuclei), CD8 (white, cytotoxic T cells) and CD31 (red, endothelium). The images show more T cells infiltrate into the middle of the tumour parenchyma in the Blockmir CD5-2 treated mice (right panel) compared to that in the control treated mice (left panel). The label miRNA refers to Blockmir CD5-2. The distance between leading edge of invasive CD8+ T cells and edge of tumour section was quantified. Data represents mean±SEM. **, P<0.01, n=5 mice per group, two independent experiments, paired t-test. (C) Representative confocal images of B16F10 melanoma sections stained for TUNEL (green) to visualize the apoptotic cells. The percentage of TUNEL positive cells was quantified. Data represents mean±SEM. n=6 mice per group, paired t-test. (D) Growth curve of primary B16F10 tumours given 30 mg/kg of control or CD5-2 intravenously into nude mice. Data represents mean±SEM, 7 mice from three independent experiments, paired t-test. (E) Representative images of pericyte coverage in nude mice treated with control or CD5-2. Pericyte coverage was quantified as previously. Data represents mean±SEM. *, P<0.05, n=3 mice per group, three independent experiments, paired t-test. (F) Representative images of smooth muscle cell coverage in nude mice treated with control or CD5-2. Smooth muscle cell coverage was quantified as previously. Data represents mean±SEM. *, P<0.05, n=3 mice per group, three independent experiments, paired t-test.

FIG. 3. Blockmir CD5-2 reduces average tumour vessel volume. (A) Representative confocal images of B16F10 melanoma sections (day 5 following the injection of control or CD5-2) stained for CD31 (red) to visualize tumour vessels in the treatment of control or Blockmir CD5-2, at 6-8 days of growth. (B) Quantification of average vessel volume. Data represents mean±SD. **, P<0.01, n=5 mice per group, paired t-test. (C) Quantification of the number of vessels per field. Data represents mean±SD. *, P<0.05, n=5 mice per group, paired t-test. 50 μm sections were taken for the analysis.

FIG. 4. SEM micrographs of tumour vessels in the treatment of control or Blockmir CD5-2. Left panel: mice were given control Blockmir. Abnormal tumour vessel containing multilayers of disconnected endothelial cells with luminal protrusions in tumour. Right panel: mice were given Blockmir CD5-2. Vessel lined by monolayer of cobblestone endothelial cells. B16F10 tumours at Day 12. Representative of 4 vessels imaged for control and 4 for CD5-2 treated mice.

FIG. 5. Blockmir CD5-2 induces pericyte and smooth muscle cell coverage in B16F10 melanoma model. (A) Endothelium and associated pericytes were visualised by CD31 (red) and NG2 (green) immunofluorescence staining respectively of B16F10 tumours from control or Blockmir CD5-2 treated mice. Bottom row, high magnification of selected area in top row. (B) Pericyte coverage was quantified by calculating the percent fraction of vessel length that overlapped with NG2 staining in the image of the vessels to determine direct contact between the two cell types. Data represents mean±SEM. *, P<0.05, n=8 mice per group, paired t-test. (C) Endothelium and associated smooth muscle cells were visualised by CD31 (red) or αSMC (green) immunofluorescence staining respectively of B16F10 tumour implants. Bottom row, high magnification of selected area in top row. (D) Smooth muscle cell coverage was quantified by calculating the percent fraction of vessel length that overlapped with αSMC staining in the image of the vessels to determine direct contact between the two cell types. Data represents mean±SEM. *, P<0.05, n=8 mice per group, paired t-test.

FIG. 6. Effects of Blockmir CD5-2 on VE-Cadherin expression in B16F10 melanoma tumour vessels. (A) Representative images of VE-Cadherin expression (green) in tumour vessels stained for CD31 (red). Top image=control Blockmir, bottom image representative image of Blockmir CD5-2 treated tumours. (B) The ratio of the fluorescence intensity of VE-Cadherin to CD31 was determined and is presented as relative values. Data represents mean±SD. n=5 mice per group. *, P<0.05, paired t-test.

FIG. 7. Blockmir CD5-2 promotes basement membrane support in the B16F10 melanoma model. (A) Endothelium and associated basement membrane were visualised by CD31 (red) and Collagen IV (green) immunofluorescence staining respectively of B16F10 tumours from mice treated with control or Blockmir CD5-2. (B) Basement membrane support of tumour vasculature was quantified by calculating the percent fraction of vessel length that overlapped with Collagen IV staining in the image of the vessels to determine direct contact between endothelial cells and basement membrane. Data represents mean±SD. **, P<0.01, n=5 mice per group, paired t-test.

FIG. 8. Blockmir CD5-2 decreases tumour vascular permeability in B16F10 melanoma model. (A) Representative images of tumour vessel leakiness in the tumours from mice treated with control or Blockmir CD5-2. R50 fluorescent microspheres were injected intravenously into C57BL/6 mice bearing B16F10 tumours. The extravasated 50 nm fluorescent microspheres (green) from tumour vessels stained for CD31 (red) are shown. (B) To quantify the level of microsphere leakage and standardize it for tumour vessel area, the ratio of the number of microspheres to CD31 area was determined and is presented as relative values. Data represents mean±SD. **, P<0.01, n=6 mice per group, paired t-test. (C) Representative images of fibrinogen deposition in the treatment of control or CD5-2 are shown. (D) To quantify the level of fibrinogen deposition and standardize it for tumour vessel area, the ratio of the area of fibrinogen to vessel was determined and is presented as relative values. Data represents mean±SEM. *, P<0.05, n=6 mice per group, paired t-test.

FIG. 9. Blockmir CD5-2 promotes tumour vascular perfusion in B16F10 melanoma model. (A) Representative images of tumour vascular perfusion in mice treated with control or Blockmir CD5-2. FITC-conjugated lectin was injected intravenously into C57BL/6 mice bearing B16F10 tumours. Double positive staining for FITC-conjugated lectin (green) and CD31 (red) was used to evaluate the perfused tumour vessels. (B) To quantify the percentage of perfused vessels (yellow), the ratio of the number of perfused vessels to total vessels was determined and is presented as relative values. Data represents mean±SD. *, P<0.05, n=6 mice per group, paired t-test.

FIG. 10. Blockmir CD5-2 diminishes tumour hypoxia in the B16F10 melanoma model. (A) Representative images of tumour hypoxia in the tumours from mice treated with control or Blockmir CD5-2. Hypoxia probe Hypoxyprobe-1 was injected intravenously into C57BL/6 mice bearing B16F10 tumours. Double positive staining for pimonidazole (green) and CD31 (red) was used to evaluate the level of tumour hypoxia. (B) Quantification of tumour hypoxic area (green) in the presence of control or Blockmir CD5-2. Data represents mean±SD. *, P<0.05, n=6 mice per group, paired t-test.

FIG. 11. Blockmir CD5-2 promotes vascular perfusion in RIP-TAG5 tumour model. (A) Representative images of tumour vascular perfusion in the treatment of control (Ctrl) or Blockmir CD5-2 (miRNA). FITC-conjugated lectin was injected intravenously into 27-week old RIP-TAG5 mice. Double positive staining for FITC-conjugated lectin (green) and CD31 (red) was used to evaluate the perfused vessels. (B) To quantify the percentage of perfused vessels (yellow), the ratio of the number of perfused vessels to total vessels was determined and is presented as relative values. Data represents mean±SEM. *, P<0.05, n=7 mice treated with control and 3 mice treated with Blockmir CD5-2, unpaired t-test.

FIG. 12. CD5-2 enhances immunotherapeutic effects. (A) Representative images of tumour vascular perfusion in RIP-Tag5 pancreatic tumours. Double positive staining for FITC-conjugated lectin (green) and CD31 (red) was used to evaluate the perfused tumour vessels. To quantify the percentage of perfused vessels (yellow), the ratio of the number of perfused vessels to total vessels was determined and is presented as relative values. Data represents mean±SEM. *, P<0.05, n=3-5 mice per group, unpaired t-test. (B) Adoptive transfer of CD8+ T cells in RIP-Tag5 pancreatic tumours. CD8+ surface area (%) was quantified. Data represents mean±SEM. *, P<0.05, n=3-5 mice per group, unpaired t-test. (C) Growth curve of MC38 tumours. Anti-PD-1 or control IgG given IP on day 7, 11, 14. Control or CD5-2 was given i.v. on day 7. Data represents mean±SEM. ***, P<0.001; **, P<0.01; *, P<0.05, n=8 mice per group, two-way ANOVA test. (D) Growth curve of MC38 tumours. Anti-PD-1 or control IgG given i.p. on day 7, 11, 14. Control or CD5-2 was given i.v. on day 6, 9, 12. Data represents mean±SEM. ***, P<0.001; **, P<0.01; *, P<0.05, n=8 mice per group, two-way ANOVA test. (E) CD8/Gr1 hi ratio of MC38 tumours with single injection of CD5-2. Data represents mean±SEM. *, P<0.05; **, P<0.01, n=7-8 mice per group, unpaired t-test. (F) The percentage of CD8+ T cells that are Granzyme B positive. Data represents mean±SEM. *, P<0.05; **, P<0.01, n=7-8 mice per group, unpaired t-test.

FIG. 13. Effects of Blockmir CD5-2 on metastasis of B16F10 melanoma. 2×10⁵ B16F10-luciferase cells were injected via tail vein into albino C57BL/6 mice. Control or Blockmir CD5-2 was intravenously injected into mice two days before and two days after the cell injection. (A) Bioluminescent imaging of metastasis 12 days following the cell injection. (B) Bioluminescent imaging of metastasis 15 days following the cell injection. (C) Bioluminescent imaging of metastasis 18 days following the cell injection. (D) Quantification of bioluminescence on day 18. Data represents mean±SD, 3 experiments.

FIG. 14. Bioluminescent images of lung and liver metastasis. Lungs and livers of mice injected with B16F10 melanoma and treated with control or Blockmir CD5-2 and harvested 18 days following the cell injection.

The subject specification contains amino acid and nucleotide sequence information prepared using the programme PatentIn Version 3.4, presented herein in a Sequence Listing. Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. Specifically, the miR-27a target ‘anti-seed’ region in the 3′UTR of VE-cadherin (CDH5) is shown in SEQ ID NO: 1. The region of the 3′UTR of human VE-cadherin containing the miR-27a ‘anti-seed’ region is shown in SEQ ID NO: 2. SEQ ID NOs: 3 to 8 show the sequences of exemplary oligonucleotides. The mature sequence of the human miR-27a (hsa_miR-27a) is shown in SEQ ID NO: 9.

### DETAILED DESCRIPTION

The articles “a” and “an” are used herein to refer to one or to more than one (i.e., to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “oligonucleotide” refers to a single-stranded sequence of ribonucleotide or deoxyribonucleotide bases, known analogues of natural nucleotides, or mixtures thereof. An “oligonucleotide” comprises a nucleic-acid based molecule including DNA, RNA, PNA, LNA, UNA or any combination thereof. An oligonucleotide that predominantly comprises ribonucleotide bases, natural or non-natural, may be referred to as an RNA oligonucleotide. Oligonucleotides are typically short (for example less than 50 nucleotides in length) sequences that may be prepared by any suitable method, including, for example, direct chemical synthesis or cloning and restriction of appropriate sequences.

“Antisense oligonucleotides” are oligonucleotides complementary to a specific DNA or RNA sequence. Typically in the context of the present invention an antisense oligonucleotide is an RNA oligonucleotide complementary to a specific mRNA or miRNA. The antisense oligonucleotide binds to and silences or represses, partially of fully, the activity of its complementary miRNA. Not all bases in an antisense oligonucleotide need be complementary to the ‘target’ or miRNA sequence; the oligonucleotide need only contain sufficient complementary bases to enable the oligonucleotide to recognise the target. An oligonucleotide may also include additional bases. The antisense oligonucleotide sequence may be an unmodified ribonucleotide sequence or may be chemically modified or conjugated by a variety of means as described herein.

The term “polynucleotide” as used herein refers to a single- or double-stranded polymer of deoxyribonucleotide, ribonucleotide bases or known analogues of natural nucleotides, or mixtures thereof. A “polynucleotide” comprises a nucleic-acid based molecule including DNA, RNA, PNA, LNA, UNA or any combination thereof. The term includes reference to the specified sequence as well as to the sequence complimentary thereto, unless otherwise indicated. Polynucleotides may be chemically modified by a variety of means known to those skilled in the art. Thus a “polynucleotide” comprises a nucleic-acid based molecule including DNA, RNA, PNA, LNA, UNA or any combination thereof.

As used herein in relation to oligonucleotides and polynucleotides, the term “nucleotide” refers to a single nucleobase or monomer unit within the oligonucleotide or polynucleotide. The terms “nucleotide” and “monomer” may be used interchangeably herein. The nucleobase may be part of a DNA, RNA, INA, LNA, UNA or combination of any two or more thereof) oligonucleotide or polynucleotide. In some embodiments, the nucleobase may be a universal base. Modified nucleobases are also contemplated by the present invention, as described hereinbelow.

The term “variant” as used herein refers to substantially similar sequences. Generally, polypeptide sequence variants also possess qualitative biological activity in common, such as receptor binding activity. Further, these polypeptide sequence variants may share at least 50%, 55%, 60%, 65%, 70%, 75%, 80%, 85%, 90%, 91%, 92%, 93%, 94%, 95%, 96%, 97%, 98% or 99% sequence identity. The term “sequence identity or “percentage of sequence identity” may be determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences.

The term “complementary” as used herein refers to the ability of two single-stranded nucleotide sequences to base pair, typically according to the Watson-Crick base pairing rules, that is, between G and C and between A and T or U. In some embodiments, G also pairs to U and vice versa to form a so-called wobble base pair. In another embodiment, the base inosine (I) may be included within an oligonucleotide of the invention. I base pairs to A, C and U. In still another embodiment, universal bases may be used. Universal bases can typically base pair to G, C, A, U and T. Often universal bases do not form hydrogen bonds with the opposing base on the other strand. In still another embodiment, a complementary sequence refers to a contiguous sequence exclusively of Watson-Crick base pairs. For two nucleotide molecules to be complementary they need not display 100% complementarity across the base pairing regions, but rather there must be sufficient complementarity to enable base pairing to occur. Thus a degree of mismatching between the sequences may be tolerated and the sequences may still be complementary. As used herein, the term “capable of base pairing with” is used interchangeably with “complementary to”.

The term “substitution” as used herein refers to a nucleobase at a particular position within an oligonucleotide or polynucleotide having been substituted for another nucleobase. The substitution may be, for example, because of the presence of a single nucleotide polymorphism in the target RNA. The term substitution also encompasses deletions of nucleobases and additions of nucleobases.

The term “Blockmir” as used herein refers to a steric blocking oligonucleotide that binds to an RNA target blocking the ability of one or more miRNA species from binding to, and affecting the activity of, said target. Blockmirs are constructed so as to be incapable of recruiting cellular RNAi machinery or RNase H. RNAi machinery refers to the cellular components necessary for the activity of siRNAs and miRNAs or for the RNAi pathway. A major component of the RNAi machinery is the RNA induced silencing complex (the RISC complex). Blockmirs are described, for example, in WO 2008/061537, WO 2012/069059 and WO 2014/053014, the disclosures of which are incorporated herein by reference.

In the context of this specification, the term “activity” as it pertains to a polynucleotide (e.g. a DNA, mRNA or miRNA), protein or polypeptide means any one or more cellular function, action, effect or influence exerted by the polynucleotide, protein or polypeptide. For example, in the context of a mRNA, activity will typically refer to expression of the mRNA, i.e. translation into a protein or peptide. Thus, regulation of the activity of a target mRNA by an oligonucleotide as described herein may include degradation of the mRNA and/or translational regulation. Regulation of mRNA activity may also include affecting intracellular transport of the mRNA.

The term “inhibiting” and variations thereof such as “inhibition” and “inhibits” as used herein do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably.

The terms “promoting” and “inducing”, and variations thereof such as “promotion” and “inducement”, as used herein do not necessarily imply the complete promotion or inducement of the specified event, activity or function. Rather, the promotion or inducement may be to an extent, and/or for a time, sufficient to produce the desired effect. The promotion or inducement of angiogenesis by oligonucleotides of the invention may be direct or indirect and may be in magnitude and/or be temporal in nature.

As used herein the term “effective amount” includes within its meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of some disorders, methods of the present invention involve “treating” the disorder in terms of reducing or ameliorating the occurrence of a highly undesirable event associated with the disorder or an irreversible outcome of the progression of the disorder but may not of itself prevent the initial occurrence of the event or outcome. Accordingly, treatment includes amelioration of the symptoms of a particular disorder or preventing or otherwise reducing the risk of developing a particular disorder.

As used herein the term “sensitivity” is used in its broadest context to refer to the ability of a cell to survive exposure to an agent designed to inhibit the growth of the cell, kill the cell or inhibit one or more cellular functions.

The term “subject” as used herein refers to mammals and includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

As described and exemplified herein the inventors have identified methods for sensitising tumours to therapy and for the modulation of tumour metastasis and/or vasculature, comprising administration of an oligonucleotide capable of binding to the sequence CUGUGA blocking the ability of a miRNA (such as miRNA miR-27a) to bind to said sequence and thereby inhibiting the miRNA from affecting the activity or expression of a polynucleotide comprising said sequence. Typically the sequence is present in the 3′UTR of the VE-cadherin mRNA. In particular embodiments administration of the oligonucleotide sensitises the tumour to immunotherapeutic, chemotherapeutic or radiotherapeutic treatments.

One aspect of the present invention provides a method for increasing the sensitivity of a tumour to immunotherapy, chemotherapy or radiotherapy, wherein the method comprises administration to a subject in need thereof of an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

Other aspects of the invention provide methods for modulating tumour metastasis, for normalizing tumour vasculature, for normalizing vessel function in a tumour and/or for promoting cell death of cells in a tumour, wherein the methods comprise exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

By way of example, normalising the tumour vasculature and/or improving vessel function may comprise or be characterized by one or more of: a change in morphology of endothelial cells, change in VE-cadherin expression, selective loss of large vessels; increase in number of small vessels, increased pericyte coverage of vessels, altered collagen IV coverage of vessels, reduced vessel permeability, reduced vessel hypoxia, increased vessel perfusion, and enhanced infiltration of immune cells, typically lymphocytes, including CD8+ T cells, CD4+ T cells and/or NK cells.

Typically, cell death can occur by programmed cell death, such as by apoptosis and can include cell changes such as membrane blebbing, cell shrinkage, nuclear and/or DNA fragmentation and condensation of chromatin.

### Oligonucleotides

Typically, the miRNA comprising the seed sequence UCACAG is miR-27a. The nucleotide sequence of mature human miR-27a (hsa-miR-27a) is provided in SEQ ID NO: 9. Additional sequence information for the miR-27a miRNA can be found at http://microrna.sanqer.ac.uk/sequences/index.shtml. Also contemplated herein are variants of this miRNA. Variants include nucleotide sequences that are substantially similar to the sequence of miR-27a. For example, a variant miRNA may comprise a sequence displaying at least 80%, 85%, 90%, 91%, 92%, 93%, 94%, 95%, 96%, 97%, 98% or 99% sequence identity to SEQ ID NO:9.

Oligonucleotides for use in accordance with the present invention typically comprise a contiguous sequence complementary to a sequence selected from the group consisting of at least about 9 contiguous bases, at least about 10 contiguous bases, at least about 11 contiguous bases, at least about 12 contiguous bases, at least about 13 contiguous bases, at least about 14 contiguous bases, at least about 15 contiguous bases, at least about 16 contiguous bases, at least about 17 contiguous bases, at least about 18 contiguous bases, at least about 19 contiguous bases, at least about 20 contiguous bases, at least about 22 contiguous bases, at least about 25 contiguous bases, at least about 30 contiguous bases, and at least about 35 contiguous bases of the sequence set forth in SEQ ID NO: 2 or the sequence of SEQ ID NO: 2 comprising 1, 2 or 3 substitutions.

In an embodiment, the oligonucleotide may comprise a contiguous sequence complementary to a sequence selected from the group consisting of no more than 8 contiguous bases, no more than 9 contiguous bases, no more than 10 contiguous bases, no more than 11 contiguous bases, no more than 12 contiguous bases, no more than 13 contiguous bases, no more than 14 contiguous bases, no more than 15 contiguous bases, no more than 16 contiguous bases, no more than 17 contiguous bases, no more than 18 contiguous bases, no more than 19 contiguous bases, no more than 20 contiguous bases, no more than 22 contiguous bases, no more than 25 contiguous bases, no more than 30 contiguous bases, and no more than 35 contiguous bases of the sequence set forth in SEQ ID NO: 2 or the sequence of SEQ ID NO: 2 comprising 1, 2 or 3 substitutions.

In another embodiment, the oligonucleotide may comprise a contiguous sequence complementary to a sequence selected from the group consisting of 8 contiguous bases, 9 contiguous bases, 10 contiguous bases, 11 contiguous bases, 12 contiguous bases, 13 contiguous bases, 14 contiguous bases, 15 contiguous bases, 16 contiguous bases, 17 contiguous bases, 18 contiguous bases, 19 contiguous bases, 20 contiguous bases, 21 contiguous bases, 22 contiguous bases, 23 contiguous bases, 24 contiguous bases, 25 contiguous bases, 30 contiguous bases, and 35 contiguous bases of the sequence set forth in SEQ ID NO: 2 or the sequence of SEQ ID NO: 2 comprising 1, 2 or 3 substitutions.

Typically the oligonucleotide binds to positions 22-27 of SEQ ID NO: 2, this region representing the complement of the seed sequence of miR-27a, being the target site for miR-27a binding to the 3′UTR of the VE-cadherin mRNA (the ‘anti-seed’ region). Base pairing between the oligonucleotide and SEQ ID NO: 2 may include positions 8-28, 8-27, 9-27, 10-27, 11-27, 12-27, 13-27, 14-27, 15-27, 16-27, 17-27, 18-27, 19-27, 20-27, 21-27, 9-28, 10-28, 11-28, 12-28, 13-28, 14-28, 15-28, 16-28, 17-28, 18-28, 19-28, 20-28 or 21-28 of SEQ ID NO: 2.

In one embodiment, base pairing between the oligonucleotide and the sequence of SEQ ID NO: 2 ends at position 27 of SEQ ID NO: 2. In other embodiments, base pairing may end at position 28, 29, 30, 31, 32 or 33 of SEQ ID NO: 2. In another embodiment, base pairing between the oligonucleotide and the sequence of SEQ ID NO: 2 begins at position 22 of SEQ ID NO: 2. In other embodiments, base pairing may start at position 21, 20, 19, 18, 17, 16, 15, 14, 13, 12, 11, 10, 9, 8, 7, 6 or 5 of SEQ ID NO: 2.

Those skilled in the art will appreciate that oligonucleotides for use in accordance with the invention may be of any suitable length depending on the precise function or use of the oligonucleotide. Typically, the oligonucleotides are between 8 and 25 bases in lengths. Even more typically, the oligonucleotides are between 10 and 20 bases in length.

For strong binding to its target RNA, the length of the oligonucleotide may be increased. In some cases, delivery into cells may be improved may using shorter oligonucleotides. Further, in other cases, the position of the oligonucleotide respective to the anti-seed sequence of the target RNA may be adjusted. For example, the position of bases complementary to position 22-27 of the target RNA of SEQ ID NO: 2 may be adjusted such that they are placed for example at the 5′end of the oligonucleotide, at the 3′end of the oligonucleotide or in or towards the middle of the oligonucleotide. Typically, the position of bases complementary to positions 22-27 are placed in the oligonucleotide such that they start at position 1, position 2, position 3, position 4, position 5 or position 6, or at a position upstream of position 2, position 3, position 4, position 5 or position 6 or at a position downstream of position 1, position 2, position 3, position 4, position 5 or position 6, wherein the positions are counted from the 5′end of the oligonucleotide.

In some embodiments, the target RNA sequence, for example the sequence of SEQ ID NO: 2 may comprise 1, 2 or 3 substitutions. Alternatively, the sequence may comprise no substitutions. Where substitutions are present, these may be located in the region of complementarity between the oligonucleotide and the target RNA. Substitutions may be single nucleotide polymorphisms (SNPs) that may enhance or decrease miRNA regulation of the given target RNA. An SNP may create a new miRNA target site so as to cause aberrant miRNA regulation of the given target RNA. RNA editing may also give rise to substitutions.

Oligonucleotides for use in accordance with the invention may be capable of activating RNase H. RNase H cleaves the RNA part of a RNA-DNA duplex and the structural requirements for RNase H activation are well-known to the skilled addressee. Similarly, oligonucleotides of the invention may be capable of recruiting the cellular RNAi machinery and directing the RNAi machinery to the target RNA. This may result in cleavage of the target RNA or translational repression of the target RNA.

However in particular embodiments of the present invention, the oligonucleotides can neither recruit the RNAi machinery nor RNase H. Thus typically, oligonucleotides of the invention are capable of blocking the activity of the RNAi machinery at a particular target RNA. The oligonucleotides may do so by sequestering the target sequence (the miRNA binding site) of the target RNA, such that the RNAi machinery will not recognize the target sequence. Oligonucleotides of the invention with this activity may also be referred to as Blockmirs, because they block the regulatory activity of a given miRNA at a particular miRNA binding site in target RNA. To achieve the ability to prevent recruitment or activation of RNase H by oligonucleotides of the invention, the oligonucleotides typically do not comprise 5 or more contiguous DNA nucleobases.

The oligonucleotides for use in accordance with the invention may comprise a variety of sequence and structural modifications, depending on the use and function of the oligonucleotide, as will be described further below. Those skilled in the art will appreciate that the sequence and structural modifications described herein are exemplary only, and the scope of the present invention should not be limited by reference to those modifications, but rather additional modifications known to those skilled in the art may also be employed provided the oligonucleotide retains the desired function or activity.

By way of example only, the oligonucleotide sequence may be modified by the addition of one or more phosphorothioate (for example phosphoromonothioate or phosphorodithioate) linkages between residues in the sequence, or the inclusion of one or morpholine rings into the backbone. Alternative non-phosphate linkages between residues include phosphonate, hydroxlamine, hydroxylhydrazinyl, amide and carbamate linkages, methylphosphonates, phosphorothiolates, phosphoramidates or boron derivatives. The nucleotide residues present in the oligonucleotide may be naturally occurring nucleotides or may be modified nucleotides. Suitable modified nucleotides include 2′-O-methyl nucleotides, 2′-O-flouro nucleotides, 2′-O-methoxyethyl nucleotides, universal nucleobases such as 5-nitro-indole; LNA, UNA, PNA and INA nucleobases, 2′-deoxy-2′-fluoro-arabinonucleic acid (FANA) and arabinonucleic acid (ANA). The ribose sugar moiety that occurs naturally in ribonucleosides may be replaced, for example with a hexose sugar, polycyclic heteroalkyl ring, or cyclohexenyl group. Alternatively, or in addition, the oligonucleotide sequence may be conjugated to one or more suitable chemical moieties at one or both ends. For example, the oligonucleotide may be conjugated to cholesterol via a suitable linkage such as a hydroxyprolinol linkage at the 3′ end. As a further example, the oligonucleotide may be conjugated to N-acetylgalactosamine (GalNAc).

Particular modifications of interest include those that increase the affinity of the oligonucleotide for complementary sequences, i.e. increase the melting temperature of the oligonucleotide base paired to a complementary sequence, or increase the biostability of the oligonucleotide. Such modifications include 2′-O-flouro, 2′-O-methyl, 2′-O-methoxyethyl groups. The use of LNA, UNA, PNA and INA monomers are also typically employed. For shorter oligonucleotides, typically a higher percentage of affinity increasing modifications are present. If the oligonucleotide is less than 12 or 10 nucleobases in length, it may be composed entirely of affinity increasing units, e.g. LNA monomers, UNA monomers or 2′-O-methyl RNA nucleobases.

In particular embodiments, the fraction of monomers in an oligonucleotide modified at either the base or sugar relatively to the monomers not modified at either the base or sugar may be less than 99%, less than 95%, less than 90%, less than 85%, less than 75%, less than 70%, less than 65%, less than 60%, less than 50%, less than 45%, less than 40%, less than 35%, less than 30%, less than 25%, less than 20%, less than 15%, less than 10%, less than 5%, less than 1%, more than 99%, more than 95%, more than 90%, more than 85%, more than 75%, more than 70%, more than 65%, more than 60%, more than 50%, more than 45%, more than 40%, more than 35%, more than 30%, more than 25%, more than 20%, more than 15%, more than 10%, and more than 5% or more than 1%.

Lipids and/or peptides may also be conjugated to the oligonucleotides. Such conjugation may both improve bioavailability and prevent the oligonucleotide from activating RNase H and/or recruiting the RNAi machinery. Conjugation of larger bulkier moieties is typically done at the central part of the oligonucleotide, e.g. at any of the most central 5 monomers. Alternatively, at one of the bases complementary to one of position 1-6 of SEQ ID NO: 1 or one of position 22-27 of SEQ ID NO: 2. In yet another embodiment, the moiety may be conjugated at the 5′end or the 3′end of the oligonucleotide. One exemplary hydrophobic moiety is a cholesterol moiety that may be conjugated to the oligonucleotide preventing the oligonucleotide from recruiting the RNAi machinery and improving bioavailability of the oligonucleotide. For example, the cholesterol moiety may be conjugated to one or more of the nucleobases complementary to positions 22-27 of the sequence of SEQ ID NO: 2, at the 3′end of the oligonucleotide, or at the 5′end of the oligonucleotide.

Different modifications may be placed at different positions within the oligonucleotide to prevent the oligonucleotide from activating RNase H and/or being capable of recruiting the RNAi machinery.

In a particular embodiment, phosphorothioate internucleotide linkages may connect the monomers in an oligonucleotide to improve the biostability of the oligonucleotide. All linkages of the oligonucleotide may be phosphorothioate linkages. In another embodiment, the fraction of phosphorothioate linkages may be less than 95%, less than 90%, less than 85%, less than 80%, less than 75%, less than 70%, less than 65%, less than 60%, less than 50%, more than 95%, more than 90%, more than 85%, more than 80%, more than 75%, more than 70%, more than 65%, more than 60% and more than 50%.

In an embodiment, the oligonucleotide may not comprise any RNA nucleobases. This may assist in preventing the oligonucleotide from being capable of recruiting the RNAi machinery increasing biostability of the oligonucleotide. For example, the oligonucleotide may consist of LNA and DNA nucleobases and these may be connected by phosphorothioate linkages as outlined above. In alternative embodiments, the oligonucleotide does not comprise any DNA nucleobases. In alternative embodiments, the oligonucleotide does not comprise any morpholino and/or LNA nucleobases.

In an embodiment, the oligonucleotide may comprise a mix of DNA nucleobases and RNA nucleobases to prevent the oligonucleotide from activating RNase H and prevent the oligonucleotide from recruiting the RNAi machinery. For example, DNA and RNA nucleobases may be alternated along the length of the oligonucleotide, or alternatively one or more DNA nucleobases may be located adjacent one another and one or more RNA nucleobases may be located adjacent one another.

In another particular embodiment, the oligonucleotide comprises a mix of LNA monomers and 2′-O-methyl RNA nucleobases. As above, LNA and 2′-O-methyl RNA nucleobases may be alternated along the length of the oligonucleotide, or alternatively one or more LNA nucleobases may be located adjacent one another and one or more 2′-O-methyl RNA nucleobases may be located adjacent one another.

In some embodiments, the number of nucleobases present in an oligonucleotide that increase the affinity of the oligonucleotide for complementary sequences is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or at least 22 nucleobases. In some embodiments, the number of nucleobases present in a oligonucleotide that increase the affinity of the oligonucleotide for complementary sequences is 1, 2, 3, 4, 5, 6, 7, 8, 9, 10, 11, 12, 13, 14, 15, 16, 17, 18, 19, 20, 21, and 22 nucleobases.

In particular embodiments, the nucleobases that increase the affinity of the oligonucleotide for complementary sequences may be located at the flanks of the oligonucleotide, i.e. at or near either or both of the 5′ and 3′ ends of the oligonucleotide, or may be located at or near the centre of the oligonucleotide. The nucleobases that increase the affinity of the oligonucleotide for complementary sequences may also be distributed evenly across the length of the oligonucleotide.

Table 1 sets out exemplary oligonucleotide sequences for use in accordance with the present invention. In a particular exemplary embodiment, the oligonucleotide has a sequence as set forth in SEQ ID NO: 5.

TABLE 1
Oligonucleotide sequences

| Oligonucleotide Sequence (5′-3′)¹ | SEQ ID NO |
|---|---|
| TUCACAGUTGCUUCA | 3 |
| TUCACAGUTGCTTCA | 4 |
| TUCACAGUTGCTTCA (CD5-2) | 5 |
| TUCACAGUTGCUUCA (CD5-1) | 6 |
| TUCACAGUTGCTTCA (CD5-3) | 7 |

¹Single underlining represents an LNA monomer; double underlining represents a 2′ O-methyl RNA monomer; bold represents a UNA monomer

Oligonucleotides used in the present invention may be administered in accordance with the embodiments disclosed herein in the form of pharmaceutical compositions, which compositions may comprise one or more pharmaceutically acceptable carriers, excipients or diluents. Such compositions may be administered in any convenient or suitable route such as by parenteral (e.g. subcutaneous, intraarterial, intravenous, intramuscular), oral (including sublingual), nasal or topical routes. In circumstances where it is required that appropriate concentrations of the oligonucleotide are delivered directly to the site in the body to be treated, administration may be regional rather than systemic. Regional administration provides the capability of delivering very high local concentrations of the oligonucleotide to the required site and thus is suitable for achieving the desired therapeutic or preventative effect whilst avoiding exposure of other organs of the body to the compound and thereby potentially reducing side effects.

Oligonucleotides of the invention may be packaged and delivered in suitable delivery vehicles which may serve to target or deliver the oligonucleotides, and optionally one or more additional agents to the required tumour site. By way of example, the delivery vehicle may comprise liposomes, or other liposome-like compositions such as micelles (e.g. polymeric micelles), lipoprotein-based drug carriers, microparticles, nanoparticles, or dendrimers.

Liposomes may be derived from phospholipids or other lipid substances, and are formed by mono- or multi-lamellar hydrated liquid crystals dispersed in aqueous medium. Specific examples of liposomes used in administering or delivering a composition to target cells are DODMA, synthetic cholesterol, DSPC, PEG-cDMA, DLinDMA, or any other non-toxic, physiologically acceptable and metabolisable lipid capable of forming liposomes. The compositions in liposome form may contain stabilisers, preservatives and/or excipients. Methods for preparing liposomes are well known in the art, for example see Methods in Cell Biology, Volume XIV, Academic Press, New York, N.Y. (1976), p. 33 ff., the contents of which are incorporated herein by reference. Biodegradable microparticles or nanoparticles formed from, for example, polylactide (PLA), polylactide-co-glycolide (PLGA), and epsilon-caprolactone (\acute{ε}-caprolactone) may be used.

Other means of packaging and/or delivering oligonucleotides, and optionally one or more additional agents, in order to facilitate delivery to the tumour site will also be well known to those skilled in the art. By way of example only, delivery platforms may include RNA-lipoplex technologies comprising cationic lipids, fusogenic or stabilising co-lipids, and PEGylated lipids.

Any suitable amount or dose of an oligonucleotide of the invention may be administered to a subject in need in accordance with the present invention. The therapeutically effective amount for any particular subject may depend upon a variety of factors including: the tumour being treated and the severity of the tumour; the activity of the conjugate employed; the composition employed; the age, body weight, general health, sex and diet of the subject; the time of administration; the route of administration; the rate of sequestration of the molecule or agent; the duration of the treatment; drugs used in combination or coincidental with the treatment, together with other related factors well known in medicine. One skilled in the art would be able, by routine experimentation, to determine an effective, non-toxic amount of protein conjugate to be employed.

The skilled addressee will recognise that in determining an appropriate and effective dosage range for administration to humans based on the mouse studies exemplified herein, dose escalation studies would be conducted. The skilled addressee would therefore appreciate that the above mentioned doses and dosage ranges are exemplary only based on the doses administered in the mouse studies exemplified herein, and the actual dose or dosage range to be employed in humans may be varied depending on the results of such dose escalation studies. Based on the data exemplified herein, the appropriate and effective dose or dosage range to be administered to humans can be determined by routine optimisation, without undue burden or experimentation.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filter sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active agents are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active.

Tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the oligonucleotides may be incorporated into sustained-release preparations and formulations.

Embodiments of the present invention also provide kits for use in accordance with the invention. For example, kits of the invention may contain one or more Blockmirs disclosed herein, and optionally scrambled oligonucleotides for use as controls. Such kits may be used, for example, in medical or biological research activities, including investigations into neutrophil activity, vascular permeability or inflammation. Kits according to the present invention may also include other components required to use the Blockmirs, such as buffers and/or diluents. The kits typically include containers for housing the various components and instructions for using the kit components in the methods of the present invention.

### Tumour Therapies

Particular embodiments disclosed herein provide for the sensitization of tumours and tumour cells to chemotherapeutic agents, immunotherapy agents or radiotherapy using oligonucleotides as disclosed herein. The tumour or tumour cells may display resistance to the chemotherapeutic agent or immunotherapy agent in the absence of treatment with the oligonucleotide.

Thus, embodiments of the invention contemplate combination treatments, wherein administration of the oligonucleotide is in conjunction with one or more additional anti-tumour therapies. Such additional therapies may include, for example, radiotherapy, chemotherapy or immunotherapy/immune stimulation/deletion of stromal immune cells known to foster tumour growth, such as myeloid suppressor cells and regulatory T cells. Contemplated herein are synergistic combinations in which the combination treatment is effective in inhibiting growth, or reducing viability, of tumour cells, to a greater extent than either component of the combination alone. Thus, in some embodiments a synergistically effective amount of oligonucleotide and, for example, a chemotherapeutic agent or immunotherapeutic agent is administered to a subject. A synergistically effective amount refers to an amount of each component which, in combination, is effective in inhibiting growth, or reducing viability, of cancer cells, and which produces a response greater than either component alone.

For such combination therapies, each component of the combination therapy may be administered at the same time, or sequentially in any order, or at different times, so as to provide the desired effect. Alternatively, the components may be formulated together in a single dosage unit as a combination product. When administered separately, the components may be administered by the same route of administration, or different routes of administration.

Immunotherapy or immune stimulation may comprise, by way of example only, adoptive cell transfer or the administration of one or more anti-tumour or immune checkpoint antibodies, small molecules, peptides, oligonucleotides, mRNA therapeutics, bispecific/trispecific/multispecific antibodies, domain antibodies, antibody fragments thereof, other antibody-like molecules (such as nanobodies, affibodies, T and B cells, ImmTACs, Dual-Affinity Re-Targeting (DART) (antibody-like) bispecific therapeutic proteins, Anticalin (antibody-like) therapeutic proteins, Avimer (antibody-like) protein technology, anti-tumour vaccines or immune-cell modulating reagents. Adoptive cell transfer typically comprises the recovery of immune cells, typically T lymphocytes from a subject and introduction of these cells into a subject having a tumour to be treated. The cells for adoptive cell transfer may be derived from the tumour-bearing subject to be treated (autologous) or may be derived from a different subject (heterologous). Suitable antibodies for use in immunotherapy or immune stimulation may include anti-CTLA4 antibodies or anti-PD-1 antibodies. However these are provided by way of example only, and those skilled in the art will appreciate that other antibodies directed to T cells or antibodies directed to other tumour cell markers may be employed. The identity of suitable anti-tumour antibodies will depend, for example, on the nature or type of tumour to be treated. Suitable anti-tumour antibodies will be well known to those skilled in the art (see, for example, Ross et al., 2003). Cells for adoptive cell transfer and anti-tumour or immune checkpoint antibodies small molecules, peptides, oligonucleotides, mRNA therapeutics, bispecific/trispecific/multispecific antibodies, domain antibodies, antibody fragments thereof, other antibody-like molecules anti-tumour vaccines or immune-cell modulating reagents may be regarded, collectively, as immunotherapy agents.

Suitable chemotherapeutic agents may be, for example, alkylating agents (such as cyclophosphamide, oxaliplatin, carboplatin, chloambucil, mechloethamine and melphalan), antimetabolites (such as methotrexate, fludarabione and folate antagonists) or alkaloids and other antitumour agents (such as vinca alkaloids, taxanes, camptothecin, doxorubicin, daunorubicin, idarubicin and mitoxantrone). In further embodiments chemotherapeutic agents may be, for example, targeted therapies, small molecule therapies, kinase inhibitors, including but not limited to protein or lipid kinase inhibitors such as inhibitors of PI3 kinase, PIM family kinase members, receptor tyrosine kinase (RTK), Flt-3, EGFR or HER2, MEK, BRaf or an anthracyclin, a taxane, a platin, a nucleotide analog, a hormone therapeutic agent, an anti-tumour compound that has potential radiosensitising and/or chemosensitising effects, such as chloroquine; an mTOR inhibitor, an Akt or PI3-K inhibitor, a JAK inhibitor; an agent that modulates the DNA damage response mechanism and/or the stress signaling pathway, an inhibitor of p38 and/or NF-KB or a BCL-2 family inhibitor. However these are provided by way of example only, and those skilled in the art will appreciate that other chemotherapeutic agents may be employed.

In embodiments, the invention provides a method for modulating normalising tumour-associated endothelial cells, normalising tumour vasculature and/or improving vascular function in a tumour by administration of the oligonucleotides of the invention. The normalization of tumour vasculature and improvement of vessel function may be determined, assessed or measured by a number of means or parameters well known to those skilled in the art. By way of example only, normalization of tumour vasculature and improvement of vessel function may comprise or be characterized by one or more of: change in morphology of endothelial cells, change in VE-cadherin expression, selective loss of large vessels; increase in number of small vessels, increased pericyte coverage of vessels, altered collagen IV coverage of vessels, reduced vessel permeability, reduced vessel hypoxia, increased vessel perfusion, and enhanced infiltration of immune cells. In a particular embodiment the immune cells are lymphocytes. In further embodiments, lymphocytes comprise CD8+ T cells, CD4+ T cells and/or NK cells. In further embodiments, the invention provides a method for reducing tumour hypoxia.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### General Methods

### Oligonucleotides

Blockmir CD5-2 was synthesized by Mirrx Therapeutics. The sequences of oligonucleotides used in the experiments described in the following examples are provided in Table 2 and in the Sequence Listing appearing at the end of the specification.

TABLE 2
Oligonucleotide sequences

| Oligonucleotide | Sequence (5′-3′)¹ | SEQ ID NO |
|---|---|---|
| CD5-2 | TUCACAGUTGCTTCA | 5 |
| Control Blockmir | TCCAGAGATGGTUGA | 8 |

¹Single underlining represents an LNA monomer; double underlining represents a 2′ O-methyl RNA monomer.

### RIP-TAG5 Tumour Model

RIP1-Tag5 mice (C3H background) express the oncogenic simian virus (SV40) large T antigen (Tag) under the control of the rat insulin gene promoter (RIP) in pancreatic β cells, and develop spontaneous pancreatic tumours. Control Blockmir or Blockmir CD5-2 was injected systemically via the tail-vein into the 27-week old RIP-Tag5 mice at a dose of 30 mg per kg of body weight in 100 μl nuclease free water. FITC-lectin was intravenously injected into tumour-bearing mice on day 7 following the injection of control Blockmir or Blockmir CD5-2. Fifteen minutes later, mice were perfused with PBS, tissues excised, and immunofluorescent staining for CD31 and FITC-lectin was performed to evaluate the percentage of perfused tumour vessels.

### Adoptive Transfer of CD8+ T Cells

27-week old RIP-Tag5 mice received adoptive transfers of 2.5×10⁶ Tag specific activated CD8+ T cells. The mice were sacrificed at day 12 and analyzed by immunofluorescent staining for CD8+ cell infiltration.

### B16F10 Melanoma Model

B16F10 melanoma cells were cultured in DMEM containing 10% FCS, 100 U/ml penicillin, and 100 m/ml streptomycin. 4×10⁵ B16F10 melanoma cells in 200 μl 1× Dulbecco's Phosphate Buffered Saline (DPBS) (Life Technologies) were injected subcutaneously into the dorsal right flank region of female C57BL6 mice or into nude mice (6-8 weeks of age). When the tumours became palpable, control or CD5-2, dissolved in 100 μl nuclease free water was injected systemically via the tail-vein into the mice at a dose of 30 mg per kg of body weight. Tumour volumes were measured every day from day 5 following injection using a digital caliper based on the formula: V=JI×[d²×D]/6, where d is the minor tumour axis and D is the major tumour axis. The mice were sacrificed before the tumour size reached ethical limits (1000 mm³) within 3 weeks.

### Evaluation of Tumour Vascular Morphology Using Scanning Electron Microscope (SEM)

The tumour tissue was harvested from the mouse and rinsed in saline to remove blood and debris on the surface of the tissue. SEM fixative (2.5% SEM grade glutaraldehyde, 2% formaldehyde pH 7.4, 2 mM calcium chloride, 2% sucrose and 0.1M Cac buffer pH 7.4) was then taken up into a syringe and directly injected into the tumour until it was hard (needle fixation). Care was taken to keep the injecting pressure low to avoid destroying the blood vessels. The tumours were cut into smaller pieces over SEM fixative and incubated in SEM fixative for 72 h at 4° C.

### Measurement of Tumour Vascular Permeability

To evaluate tumour vascular permeability, fluorescent 50 nm polymer microspheres R50 (250 μl/kg) were diluted in 0.9% NaCl to a volume of 100 μl and injected into the tumour-bearing C57BL6 mouse via the tail vein. The fluorescent microspheres were allowed to circulate for 6 hours before the tumours were harvested, embedded in optimal cutting temperature (OCT) compound, and 8 μm frozen sections were cut in a cryostat (Leica, Germany). Specimens were examined with a confocal fluorescence microscope (Leica SP5) and quantified with Image J software (National Institute of Mental Health, MD, USA).

### Evaluation of Tumour Vascular Perfusion

To assess tumour vascular perfusion, tumour-bearing mice were injected with 150 μl of 2 mg/ml fluorescein isothiocyanate-conjugated tomato (Lycoper-siconesculentum) lectin (Vector Laboratories) diluted in 0.9% NaCl intravenously into the tail vein. After FITC-lectin was allowed to circulate for 5 min, the tumours were excised, embedded in optimal cutting temperature (OCT) compound, and 8 μm frozen sections were cut in a cryostat. The frozen tumour sections were fixed, blocked, incubated with CD31 antibody, and then incubated with Alexa 647 goat anti-rat secondary antibody. Specimens were examined with a confocal fluorescence microscope (Leica SP5) and a perfusion index was quantified as the percentage of lectin-positive vessels per CD31-positive vessel in each confocal fluorescent microscopic field.

### Assessment of Tumour Hypoxia

In order to measure tumour hypoxia, tumour-bearing mice were injected intravenously with 60 mg/kg of Hypoxyprobe-1 (HP2-100; Chemicon, Temecula, Calif.) that had been resuspended at a concentration of 30 mg/ml in 0.9% sterile saline. The solution was allowed to circulate for 90 minutes before the tumours were removed, embedded in OCT compound, and 8 μm frozen sections were cut in a cryostat. The frozen tumour sections were fixed, blocked, and incubated with rabbit anti-CD31 and mouse anti-pimonidazole (Chemicon) primary antibodies, followed by incubation with Alexa 647 goat anti-mouse and Alexa 488 goat anti-rabbit secondary antibodies using a mouse-on-mouse staining kit (Vector Laboratories, Burlingame, Calif.). Six random photographs were taken of each tissue and an average of three mice per group were used to quantify hypoxia area.

### TUNEL Staining

Cell apoptosis in OCT-embedded melanoma tissue was determined by TdT-mediated dUTP-biotin nick-end labelling (TUNEL) using in situ cell death detection kit (Roche) following the manufacturer's instructions. After blocking with 0.5% casein for 1 h, sections were incubated for 1 h at 37° C. in the dark under humidified atmosphere with 50 μl mixture of enzyme solution (TdT) and label solution (fluorescein-dUTP) at ratio of 1:25, which was followed by washed twice with PBS (5 min each) and counterstained with a 400 ng/ml solution of DAPI for 15 min in the dark. After two further washes in PBS, the sections were mounted under cover slip with mounting medium.

### Adoptive Transfers in RIP1-Tag5 Tumour Model

RIP1-Tag5 transgenic mice were bred on a C3HeBFe background. Mice transgenic for a Tag-specific T cell Receptor (TCR), restricted to H-2K^{k} (referred to as TagTCR8) were used. Tumour-bearing RIP1-Tag5 mice were treated at week 27. Adoptive transfers of in vitro activated CD8 T cells were performed as previously described (Johansson et al., 2012). Briefly, CD8+ T cells were harvested from TagTCR8 lymph nodes and spleen, and activated for 3 days in the presence of 10 U/ml IL2 (Peprotech) and 25 nM Tag peptide 560-568 (SEFLIEKRI). Tumour-bearing RIP1-Tag5 mice received a total of 2.5×10⁶ CD8+ T cells i.v. and i.p, on day 6 after miRNA injection. Tumours were analysed 4 days after adoptive therapy for tumour infiltrating lymphocytes.

### MC38 Colon Cancer Model

1×10⁶ MC38 tumour cells (in 100 μl PBS) were subcutaneously injected into mice on day 0. 250 μg of control Ig (2A3) and purified anti-mouse PD1 mAb (RMP1-14) were intraperitoneally injected into the mice on days 8, 12 and 16. 30 mg/kg control and CD5-2 were intravenously injected into the mice on day 8. Tumour growth was measured using a digital caliper and tumour size was presented as mean±SEM. The tumours were harvested from mice that had been treated with different reagents and processed for flow cytometric analysis using methods well known in the art.

### Monitoring Tumour Growth Using In Vivo Imaging System

B16F10-luc-G5 melanoma cells continuously expressing luciferase were maintained in DMEM with 10% FCS, 100 U/ml penicillin, and 100 μg/ml streptomycin at 37° C. in a humidified atmosphere of 5% CO₂. 1×10⁶ B16F10-luc-G5 melanoma cells in 200 μl× DPBS (Life Technologies) were injected subcutaneously into the dorsal right flank region of Albino B6 (C57BL/6J-Tyr<c-2J>) mice. When the tumours became palpable (generally day 9 following the injection of B16F10-luc-G5 cells), control or CD5-2 was injected systemically via the tail-vein into the mice at a dose of 30 mg per kg of body weight.

Tumour growth was monitored using the Xenogen IVIS 200 imaging system (Caliper Life Sciences) and images were taken using Living Image Software every 3 days once the presence of tumour was confirmed. Mice were anaesthetised during imaging process using isoflurane/oxygen gaseous anaesthetic (induced at 4% isoflurane and maintained at 2% isoflurane) and given intraperitoneal injections of 200 μl D-luciferin (10 μl/g body weight of 15 mg/ml stock solution, Gold Biotechnology). Each set of Albino B6 received injections within 40 seconds and in the same order. Images measuring the bioluminescent activity of the luciferase enzyme were acquired exactly at 15 min post intraperitoneal injections (3 min exposure, no time delay). The luminescent camera was set to medium binning, if/stop, blocked excitation filter, and open emission filter. The photographic camera was set to medium binning and 8f/stop. Field of view was set to E (22 cm) to image 5 mice at once. Identical settings were used to acquire each image and region of interest. Images were quantified by using LIVINGIMAGE 2.50 software.

### Monitoring Metastasis Using In Vivo Imaging System

Studies of experimental pulmonary metastasis were carried out using B16F10-luc-G5 cells that had been engineered to stably express firefly luciferase. Cells were injected intravenously into the lateral tail vein of Albino B6 (C57BL/6J-Tyr<c-2J>) mice (10-12 weeks old). Two days prior to the injections of B16F10-luc-G5 cells, control or Blockmir CD5-2 dissolved in 100 μl nuclease free water was injected via the tail-vein into the mice at a dose of 30 mg per kg of body weight. Another injection of control or Blockmir CD5-2 was performed two days after the cell injection. Metastasis was monitored using the Xenogen IVIS 200 imaging system (Caliper Life Sciences) and images were taken using Living Image Software every 3 days from day 10 following the injections of B16F10-luc-G5 cells. Mice were anaesthetised during imaging process using isoflurane/oxygen gaseous anaesthetic (induced at 4% isoflurane and maintained at 2% isoflurane) and given intraperitoneal injections of 200 μl D-luciferin (10 μl/g body weight of 15 mg/ml stock solution). The parameter settings of IVIS are described in details as above mentioned.

### Statistics

Statistical analyses using a two-tailed Student's t-test were performed with Graph-Pad Prism 5.0 (GraphPad software, San Diego, Calif., USA). Data are presented as mean±standard error (S.E.M) or mean±standard deviation (S.D.), as indicated in figure legends. Differences were considered statistically significant at P<0.05.

### Example 1—CD5-2 Facilitates Infiltration of CD8+ T Cells into Tumours and Induces Tumour Apoptosis

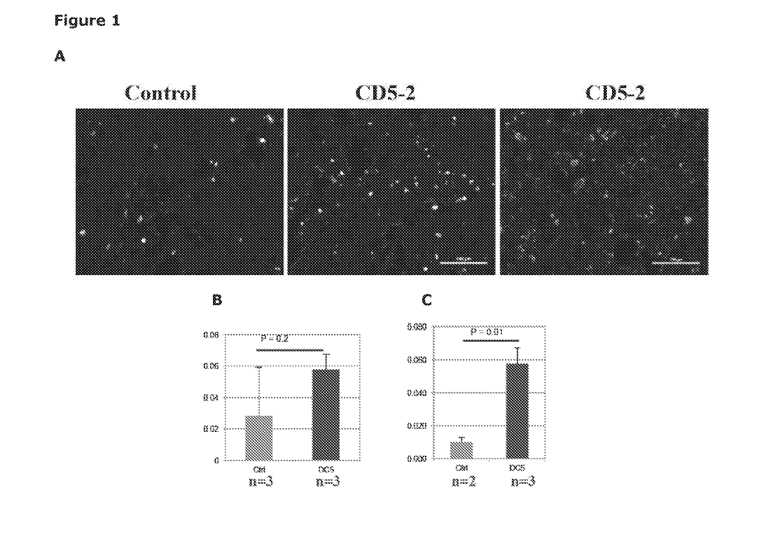

RIP1-TAG5 mice were injected with control Blockmir or Blockmir CD5-2 at 27 weeks of age. Mice were then injected with Tag specific activated CD8+ T cells 2 days later and sacrificed a further 12 days later. Infiltration of CD8+ T cells was determined by immunofluorescent staining for CD8. Mice injected with CD5-2 demonstrated an increased ratio of infiltrated T cells (relative to cells in the field of view) compared with those injected with control Blockmir (FIG. 1), indicating that Blockmir CD5-2 promotes tumour infiltration by adoptively transferred T cells. Thus CD5-2 can modulate the tumour microenvironment to increase sensitivity of solid tumours to an immune response.

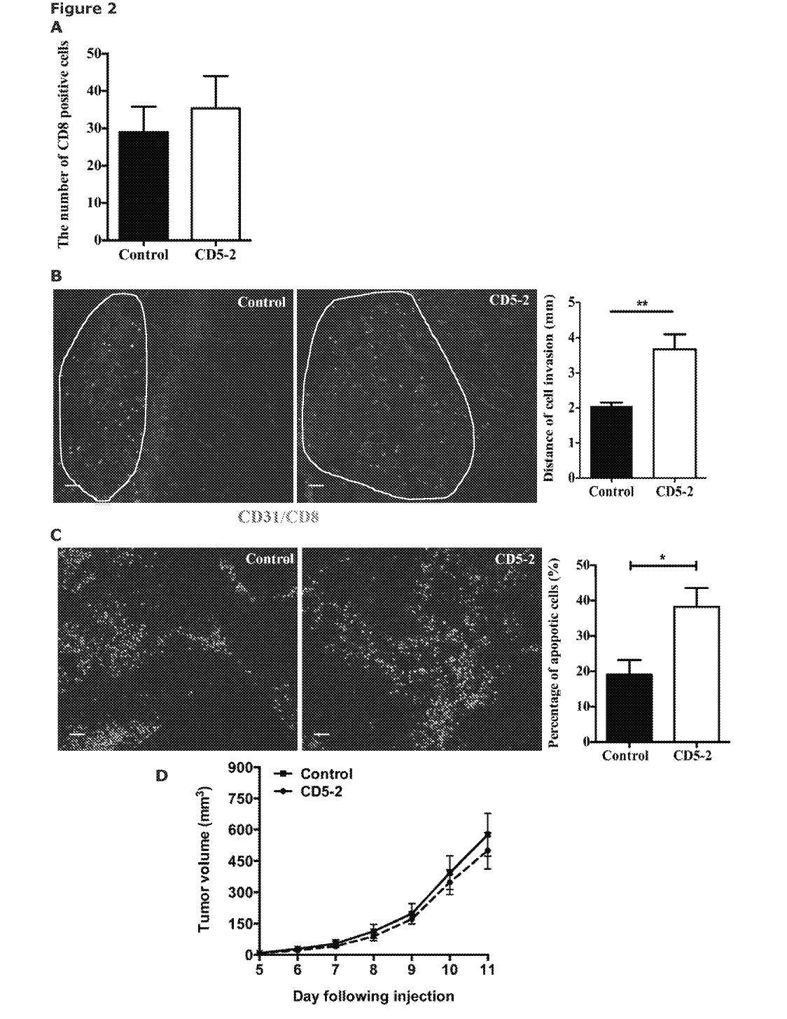

In a further model, the B16F10 melanoma model, infiltration of endogenous T cells into the middle of the tumour parenchyma is increased in Blockmir CD5-2 treated mice compared to that in the control Blockmir treated mice (FIG. 2) demonstrating that Blockmir CD5-2 may be used to facilitate immune therapies in tumour treatment. In particular, when CD8+ staining was used to evaluate the infiltration of cytotoxic T cells there was no significant difference found in the number of infiltrated CD8+ T cells in tumour parenchyma between control and CD5-2 treated groups by immunofluorescence staining (FIG. 2A). However, in 8/10 mice analysed, the CD8+ T cells in CD5-2-treated tumours were positioned more centrally in the tumour, whereas in the control treated tumours the CD8+ T cells were mainly located in the marginal area of the tumours (10/10 mice) (FIG. 2B). This effect was specific for CD8+ T cells as no significant change in the number or localisation of CD4+ T cells, CD45+ lymphocytes or in F4/80+ monocytes (data not shown) was observed.

Furthermore, CD5-2 resulted in a significant increase in apoptosis within the tumour mass as measured by TUNEL positive cells (FIG. 2C).

The position of the CD8+ T cells and the enhanced degree of apoptosis following CD5-2 treatment suggests that the retardation of tumour growth is likely to be immune mediated. To confirm this possibility the inventors used immunocompromised nude mice. CD5-2 had no effect on the tumour growth (FIG. 2D) in nude mice. However, it did alter the vasculature in these mice as both pericyte (FIG. 2E) and smooth muscle cell (FIG. 2F) coverage were enhanced following the delivery of CD5-2.

### Example 2—Blockmir CD5-2 Modulates Tumour Vasculature and Tumour Vascular Cell Morphology

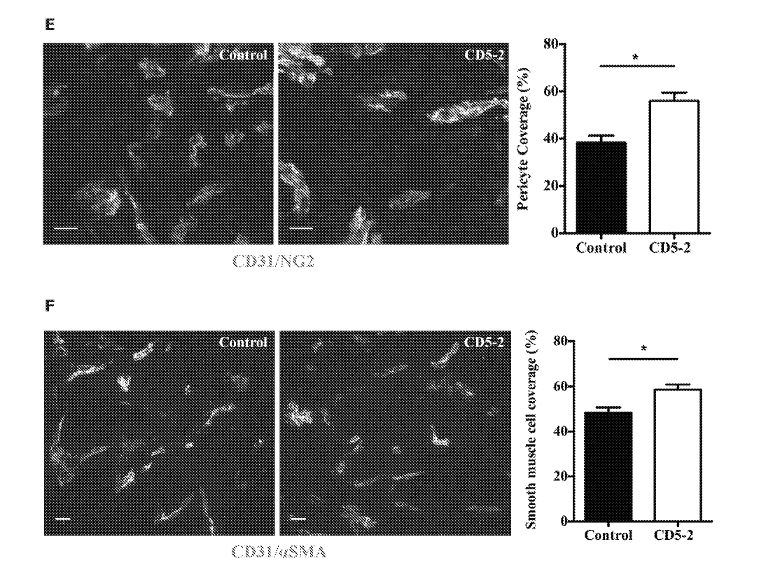

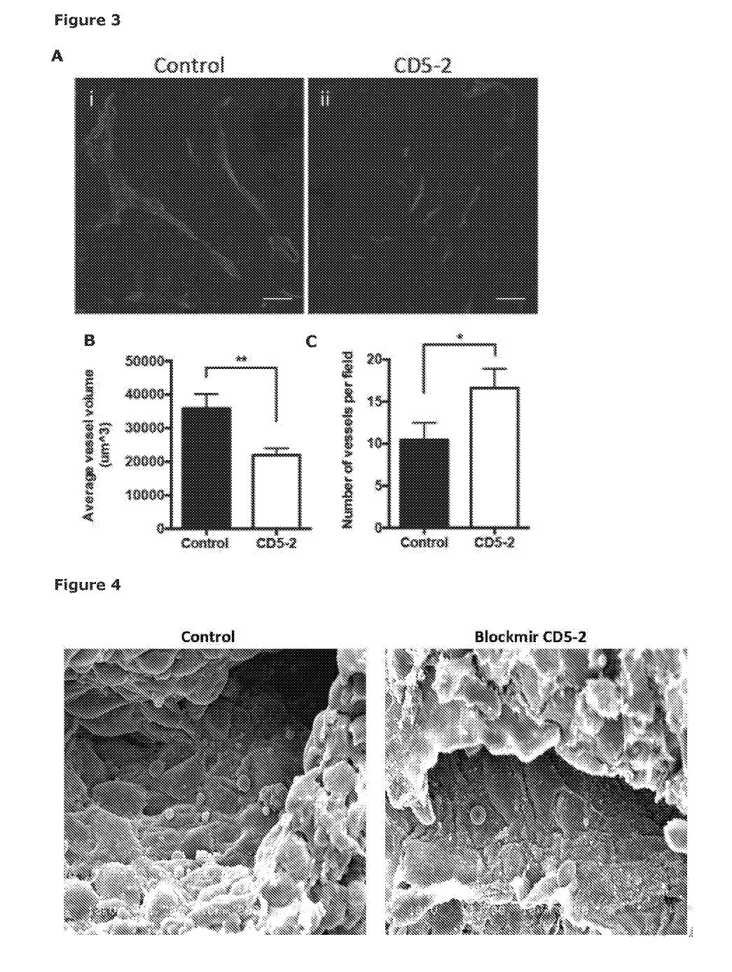

Tumour vasculature in mice with B16F10 melanoma was visualised using immunohistochemistry for CD31. Mice treated with Blockmir CD5-2 6 to 8 days prior to sacrifice displayed smaller vessels within tumours than control Blockmir treated mice. While the number of vessels per field in Blockmir CD5-2 treated mice was higher than that of control Blockmir treated mice, the average blood vessel volume was significantly reduced in mice that received Blockmir CD5-2 (FIG. 3), indicating that Blockmir CD5-2 can alter the morphology of tumour vasculature without vessel pruning. Consistent with this, CD5-2 treated ECs in vitro showed no changes in angiogenic-associated characteristics including proliferation (data not shown), senescence (data not shown) and migration (data not shown).

FIG. 4 demonstrates scanning electron microscopy of blood vessels within a B16F10 melanoma. Abnormal morphology of endothelial cells is seen in control Blockmir treated mice in which endothelial cells display properties of a non-quiescent, hyperactive endothelium appearing loosely connected and detached from each other. Endothelial cells are multi layered, rounded, disconnected and display luminal protrusions. Obvious gaps in the vessel wall are present, showing weak characteristics of cell-cell contact. Treatment with Blockmir CD5-2 normalises endothelial cell appearance of the tumour vessels as evidenced by increased organisation into a flattened single monolayer with cobblestone appearance indicative of a quiescent and less active endothelium.

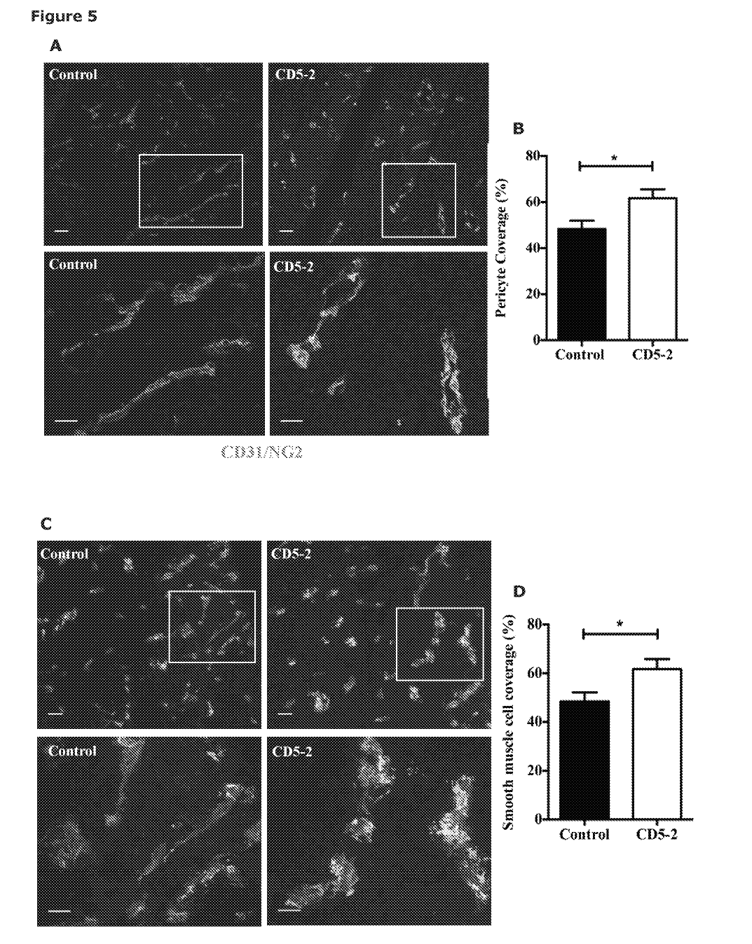

Pericyte coverage of the endothelium is also altered by Blockmir CD5-2 treatment. Pericytes are characteristically poorly attached to tumour vasculature. In the B16F10 model of melanoma, mice treated with Blockmir CD5-2 demonstrate significantly higher colocalisation of pericytes and endothelial cells than mice treated with control Blockmir (FIGS. 5A and 5B) indicating Blockmir CD5-2 helps to normalise tumour vasculature. Smooth muscle cell coverage, defined by αSMA expression, is also increased in tumour-associated vasculature following CD5-2 administration (FIGS. 5C and 5D).

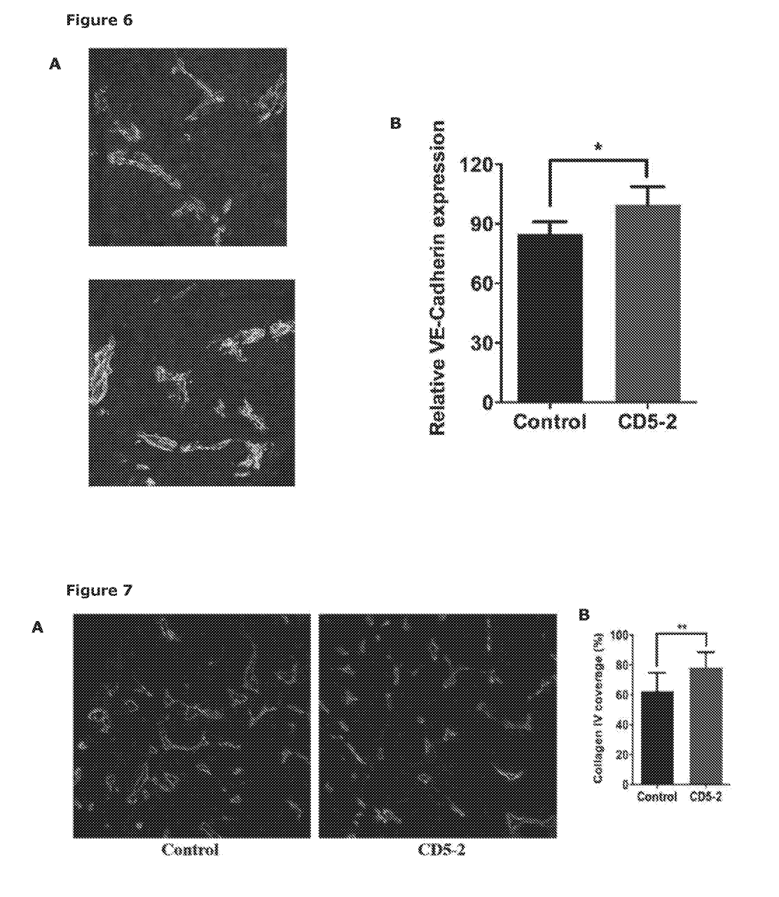

Blockmir CD5-2 also regulates expression of VE-cadherin in tumour-associated vessels, VE-cadherin being increased in the vasculature of Blockmir CD5-2 treated mice relative to control Blockmir treated mice (FIG. 6). Similarly, Blockmir CD5-2 treatment increased collagen IV coverage of tumour vessels compared to control treated animals (FIG. 7) implicating an effect of Blockmir CD5-2 on the extracellular matrix and the integrity of the basement membrane. Together, these structural changes suggest that tumour-associated vessels are “normalised” by CD5-2 treatment.

### Example 3—Tumour Vasculature Function is Altered by Blockmir CD5-2

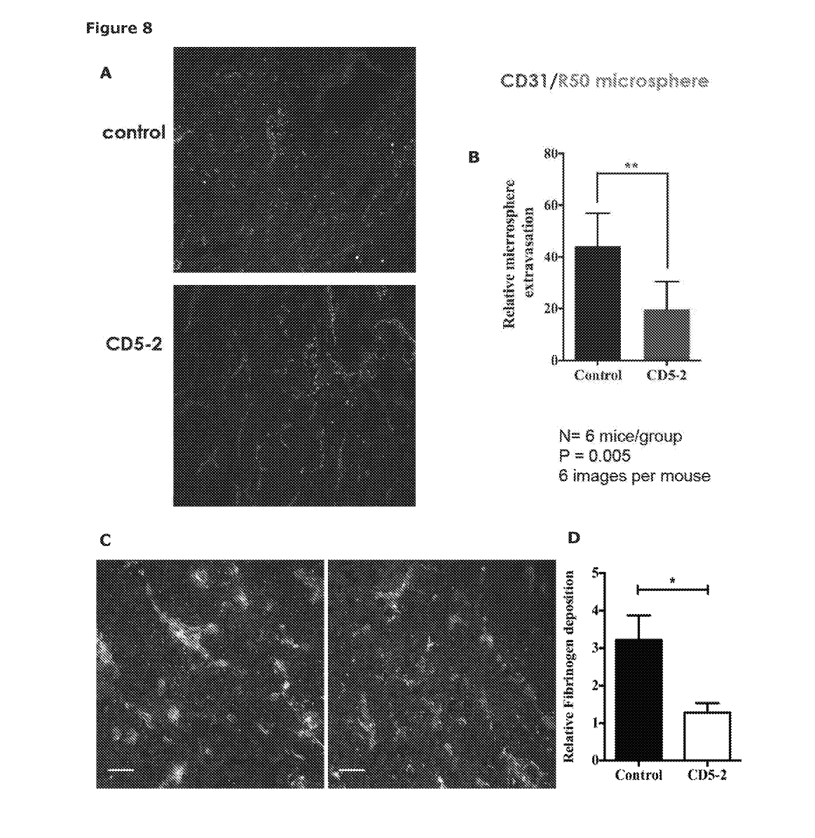

In addition to altered structure of tumour vasculature, Blockmir CD5-2 modulated permeability and perfusion of tumour vasculature in the B16F10 mouse melanoma model. FIG. 8 demonstrates the extravasion of R50 fluorescent microspheres injected into control Blockmir or Blockmir CD5-2 treated mice. CD5-2 reduced the vessel permeability as measured by the number of R50 fluorescent microspheres within the tumour parenchyma (FIG. 8A). As shown in FIG. 8B, there was a significant reduction in the ratio of microspheres to endothelial marker (CD31) in Blockmir CD5-2 treated animals relative to controls, indicating reduced leakiness of vessels with Blockmir CD5-2 treatment. Consistent with this decreased vascular permeability there was a decrease in the extent of fibrinogen deposited into the matrix (FIGS. 8C and 8D).

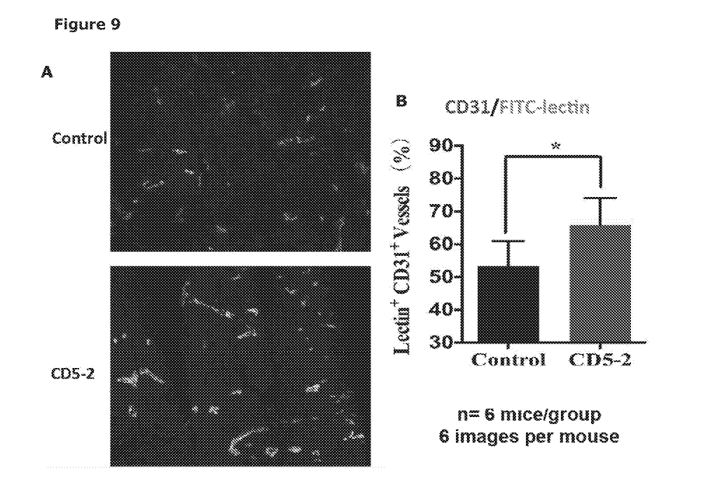

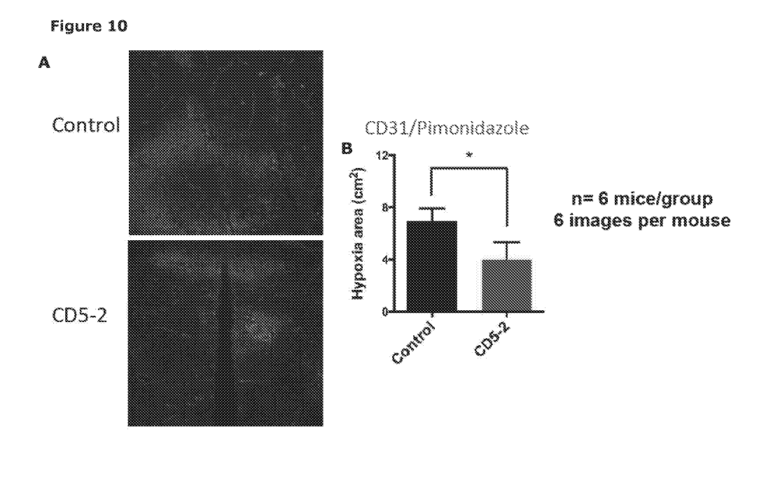

Furthermore, the percentage of perfused tumour vessels, as demonstrated with FITC-lectin/CD31 immunofluorescence colocalisation, was increased with Blockmir CD5-2 administration relative to control Blockmir treatment (FIG. 9). The hypoxic microenvironment of a tumour is chiefly related to an abnormal vessel network and the consequently abnormal perfusion. A reduction in hypoxic area of the tumour, as detected by pimonidazole staining of the hypoxia probe Hypoxyprobe-1, was also demonstrated Blockmir CD5-2 treated mice (FIG. 10) demonstrating a functional change in tumour vasculature in response to Blockmir CD5-2.

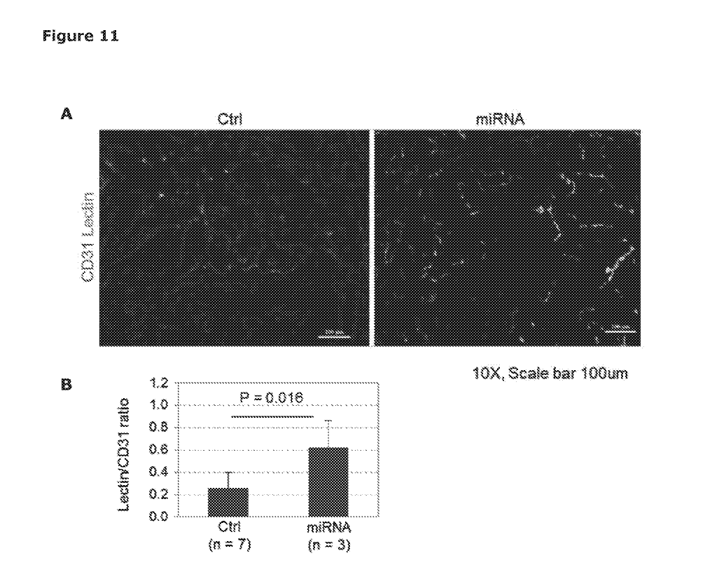

In the RIP-TAG5 mouse model, detection of FITC-lectin immunofluorescence was also increased relative to CD31 in mice treated with CD5-2 relative to those treated with a control Blockmir (FIG. 11) indicating improved perfusion of the tumour vasculature in the presence of CD5-2.

### Example 4—CD5-2 Enhances Immunotherapeutic Effects

The effects of CD5-2 to normalise the vasculature of the tumour-associated vessels and the enhancement of CD8⁺ T cells into the central regions of the microenvironment suggest that it might function to promote immunotherapy. To test this the inventors used two models, the RIP-Tag5 pancreatic neuroendocrine tumour model as a model for adaptive T cell therapy and the colon carcinoma MC38 model, which is sensitive to check-point blockade (the administration of anti-PD1 antibody).

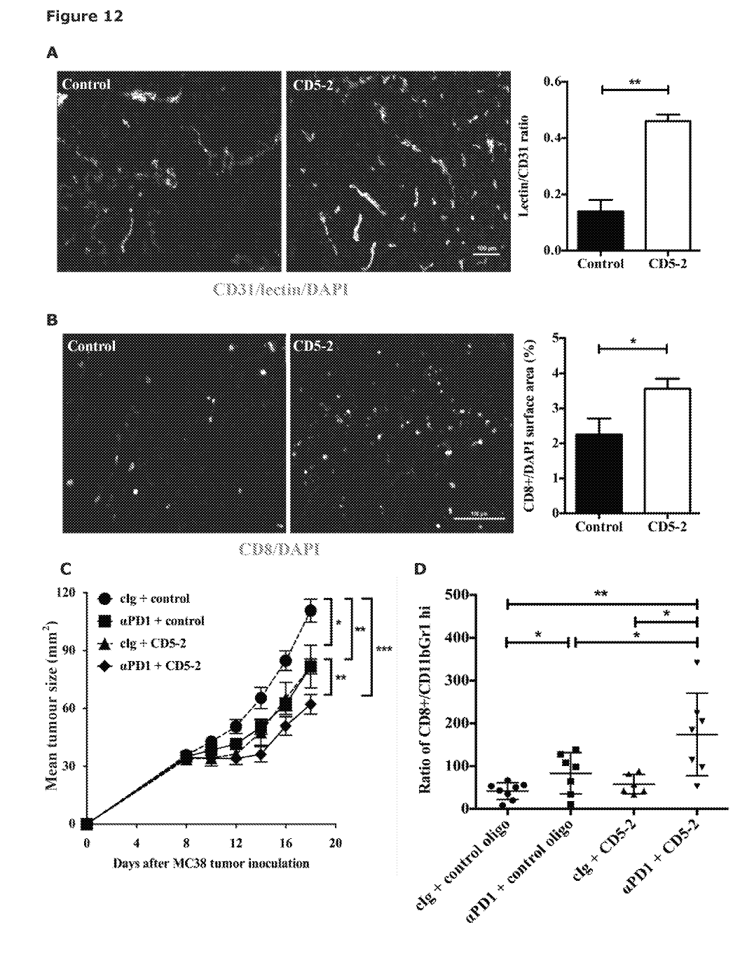

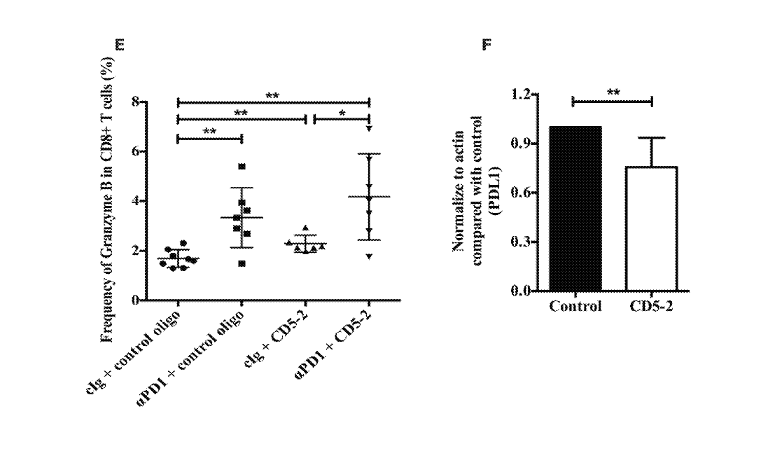

In the RIP-Tag5 model, CD5-2 enhanced the perfusion of the vessels, confirming an effect on the vasculature, similar to that seen in the B16F10 model (FIG. 12A). Tumour-specific CD8⁺ T cells were activated ex vivo and these cells were then adoptively transferred into tumour-bearing RIP-Tag mice that had previously been treated with either CD5-2 or control. CD5-2 treatment resulted in a significant enhancement in the infiltration of the activated tumour specific CD8+ T cells (FIG. 12B).

In the MC38 model, a single delivery of CD5-2 resulted in a significant inhibition of tumour growth, to similar levels as for anti-PD-1 alone (FIG. 12C). Strikingly, the combination treatment of 3 injections of anti-PD-1 and a single injection of CD5-2 resulted in a significant inhibition of tumour growth, over that seen for either treatment alone. Analysis of the immune infiltrate showed that CD5-2+ anti-PD-1 treatment resulted in a significant increase in the CD8+/CD11b/Gr1 hi ratio (FIG. 12D). Further, the percentage of CD8+ T cells that are Granzyme B positive, as an indication of activation, was increased by CD5-2 by itself and also by anti-PD-1 (FIG. 12E). CD5-2 treatment of EC monolayers in vitro results in a decrease in the PD-1 ligand, PDL1 (FIG. 12F), which may contribute to an increase in activation of CD8+ T cells in an in vivo context.

### Example 5—Blockmir CD5-2 Reduces Tumour Metastasis

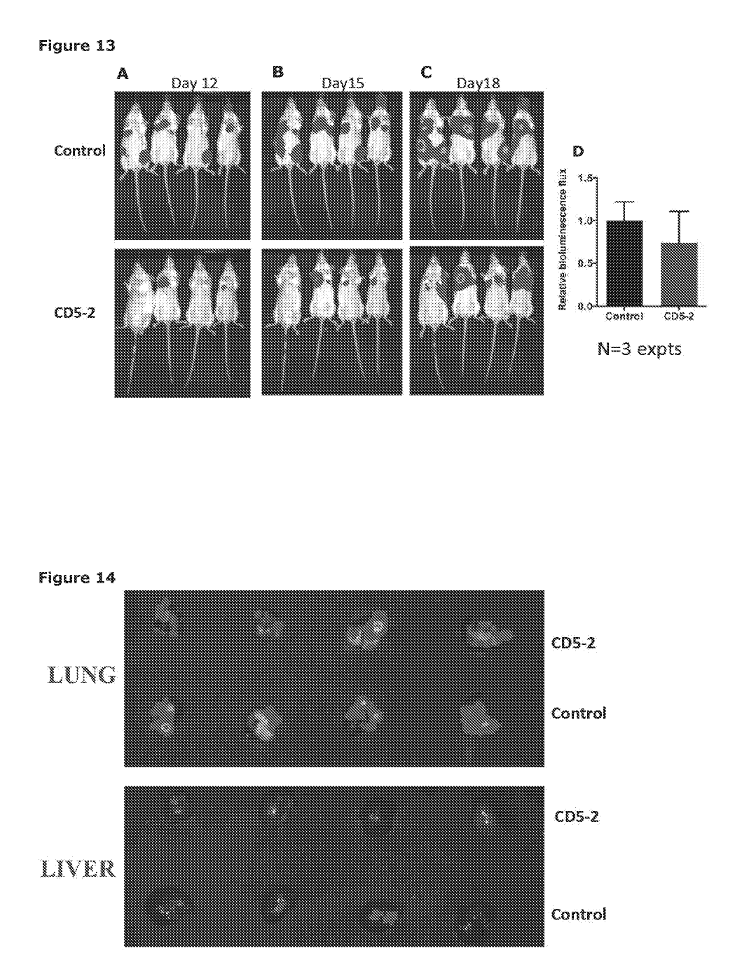

B16F10 melanoma cells expressing luciferase were injected into C57BL/6 mice and visualised with the injection of luciferin at 12, 15 or 18 days post-tumour cell injection. In mice treated with Blockmir CD5-2, bioluminescence was qualitatively reduced compared to mice injected with control Blockmir (FIG. 13). FIG. 12D demonstrates a non-significant reduction in bioluminescence at 18 days post-cell injection in mice treated with Blockmir CD5-2 relative to control Blockmir. Additionally, the lungs and livers harvested from Blockmir CD5-2 treated mice after sacrifice demonstrated qualitatively less bioluminescence than did those from control Blockmir treated mice (FIG. 14) indicating that Blockmir CD5-2 can reduce metastasis.

1. A method for increasing the sensitivity of a tumour to immunotherapy, chemotherapy or radiotherapy, wherein the method comprises administering to a subject in need thereof an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

2. The method of claim 1, wherein the method further comprises immunotherapy, chemotherapy or radiotherapy of the tumour in the subject.

3. The method of claim 2, wherein the oligonucleotide is administered to the subject prior to, concomitantly with, after, or otherwise in combination with, immunotherapy, chemotherapy or radiotherapy of the tumour.

4. The method of any one of claims 1 to 3, wherein the oligonucleotide comprises a contiguous sequence complementary to at least 8 contiguous bases of an RNA sequence comprising SEQ ID NO: 2, or SEQ ID NO: 2 comprising 1, 2 or 3 substitutions, wherein the oligonucleotide inhibits the binding of miR-27a, a variant thereof or a miRNA comprising a seed region comprising the sequence UCACAG, to said RNA.

5. The method of any one of claims 1 to 4, wherein the miR-27a miRNA is hsa-miR-27a comprising the nucleotide sequence set forth in SEQ ID NO:9.

6. The method of any one of claims 1 to 5, wherein the oligonucleotide comprises a contiguous sequence complementary to a sequence of at least or about 7 bases, at least or about 8 bases, at least or about 9 bases, at leak or about 10 bases, at least or about 11 bases, at least or about 12 bases, at least or about 13 bases, at least or about 14 bases, at least or about 15 bases, at least or about 16 bases, at least or about 17 bases, at least or about 18 bases, at least or about 19 bases, at least or about 20 bases, at least or about 22 bases, at least or about 25 bases, at least or about 30 bases, or at least or about 35 bases of SEQ ID NO: 2, or SEQ ID NO: 2 comprising 1, 2 or 3 substitutions.

7. The method of any one of claims 1 to 6, wherein the oligonucleotide binds to positions 22-27 of SEQ ID NO: 2.

8. The method of any of claims 1 to 7, wherein base pairing between the oligonucleotide and SEQ ID NO: 2 includes positions 8-28, 8-27, 9-27, 10-27, 11-27, 12-27, 13-27, 14-27, 15-27, 16-27, 17-27, 18-27, 19-27, 20-27, 21-27, 9-28, 10-28, 11-28, 12-28, 13-28, 14-28, 15-28, 16-28, 17-28, 18-28, 19-28, 20-28 or 21-28 of SEQ ID NO: 2.

9. The method of any one of claims 1 to 8, wherein the oligonucleotide comprises the sequence set forth in SEQ ID NO: 3 or SEQ ID NO: 4.

10. The method of any one of claims 1 to 9, wherein the oligonucleotide comprises one or more modified nucleobases.

11. The method of claim 10, wherein the modified nucleobase is an LNA nucleobase, a UNA nucleobase or a 2′ O-methyl nucleobase.

12. The method of any one of claims 1 to 11, wherein the oligonucleotide comprises a sequence set forth in SEQ ID NO: 5.

13. The method of any one of claims 1 to 12, wherein the immunotherapy comprises immune stimulation.

14. The method of claim 13, wherein the immune stimulation comprises adoptive cell transfer or the administration of one or more anti-tumour or immune checkpoint antibodies, small molecules, peptides, oligonucleotides mRNA therapeutics, bispecific/trispecific/multispecific antibodies, domain antibodies, antibody fragments thereof, antibody-like molecules, anti-tumour vaccines or other immune cell modulating agents.

15. The method of claim 14, wherein said adoptive cell transfer comprises the transfer of autologous tumour infiltrating lymphocytes.

16. The method of claim 14, wherein the anti-tumour antibodies comprise anti-PD-1 antibodies.

17. A method for modulating tumour metastasis, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

18. The method of claim 17, wherein said modulating tumour metastasis comprises reducing tumour metastasis.

19. A method for normalising tumour vasculature and/or improving vascular function in a tumour, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

20. The method of claim 19, wherein normalizing the tumour vasculature and/or improving vessel function comprises or is characterized by one or more of: change in morphology of endothelial cells, change in VE-cadherin expression, selective loss of large vessels; increase in number of small vessels, increased pericyte coverage of vessels, altered collagen IV coverage of vessels, reduced vessel permeability, reduced vessel hypoxia, increased vessel perfusion, and enhanced infiltration of immune cells.

21. The method of claim 20, wherein the immune cells comprise lymphocytes, neutrophils, monocytes and/or macrophages.

22. The method of claim 21, wherein lymphocytes comprise CD8+ T cells, CD4+ T cells and/or NK cells.

23. A method for reducing tumour hypoxia, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

24. A method for promoting cell death of tumour cells, the method comprising exposing a tumour to an effective amount of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA.

25. Use of an oligonucleotide comprising a contiguous sequence complementary to at least 8 contiguous bases of an RNA sequence comprising SEQ ID NO: 1, or SEQ ID NO: 1 comprising 1, 2 or 3 substitutions, wherein the oligonucleotide inhibits the binding of miR-27a, a variant thereof or a miRNA comprising a seed region comprising the sequence UCACAG, to said RNA for the manufacture of a medicament for sensitising tumours, modulating tumour metastasis, normalising tumour vasculature and/or promoting cell death of tumour cells.


SEQUENCE LISTING (9 sequences)

SEQ ID NO: 1 - RNA, 6 nt, Homo sapiens
cuguga

SEQ ID NO: 2 - RNA, 45 nt, Homo sapiens
gcuuauuaaacuuugaagcaacugugaauucauucuggaggggca

SEQ ID NO: 3 - DNA, 15 bp, Artificial
  Synthetic oligonucleotide
tucacagutgcuuca

SEQ ID NO: 4 - DNA, 15 bp, Artificial
  Synthetic oligonucleotide
tucacagutgcttca

SEQ ID NO: 5 - DNA, 15 bp, Artificial
  Synthetic oligonucleotide
  modified_base (1)..(1): LNA
  modified_base (2)..(2): 2′ O-methyl uridine
  modified_base (3)..(3): 2′ O-methyl cytidine
  modified_base (4)..(4): 2′ O-methyl adenosine
  modified_base (5)..(5): 2′ O-methyl cytidine
  modified_base (6)..(6): 2′ O-methyl adenosine
  modified_base (7)..(7): 2′ O-methyl guanosine
  modified_base (8)..(8): 2′ O-methyl uridine
  modified_base (9)..(15): LNA
tucacagutgcttca

SEQ ID NO: 6 - DNA, 15 bp, Artificial
  Synthetic oligonucleotide
  modified_base (1)..(1): LNA
  modified_base (2)..(2): 2′ O-methyl uridine
  modified_base (3)..(3): LNA
  modified_base (4)..(4): 2′ O-methyl adenosine
  modified_base (5)..(5): LNA
  modified_base (6)..(6): 2′ O-methyl adenosine
  modified_base (7)..(7): LNA
  modified_base (8)..(8): 2′ O-methyl uridine
  modified_base (9)..(9): LNA
  modified_base (10)..(10): 2′ O-methyl guanosine
  modified_base (11)..(11): 2′ O-methyl cytidine
  modified_base (12)..(12): LNA
  modified_base (13)..(13): 2′ O-methyl uridine
  modified_base (14)..(15): LNA
tucacagutgcuuca

SEQ ID NO: 7 - DNA, 15 bp, Artificial
  Synthetic oligonucleotide
  modified_base (1)..(1): LNA
  modified_base (2)..(2): 2′ O-methyl uridine
  modified_base (3)..(3): 2′ O-methyl cytidine
  modified_base (4)..(4): 2′ O-methyl adenosine
  modified_base (5)..(5): UNA
  modified_base (6)..(6): 2′ O-methyl adenosine
  modified_base (7)..(7): 2′ O-methyl guanosine
  modified_base (8)..(8): 2′ O-methyl uridine
  modified_base (9)..(15): LNA
tucacagutgcttca

SEQ ID NO: 8 - DNA, 15 bp, Artificial
  Synthetic oligonucleotide
  modified_base (1)..(1): LNA
  modified_base (2)..(2): 2′ O-methyl cytidine
  modified_base (3)..(3): LNA
  modified_base (4)..(4): 2′ O-methyl adenosine
  modified_base (5)..(5): LNA
  modified_base (6)..(6): 2′ O-methyl adenosine
  modified_base (7)..(7): LNA
  modified_base (8)..(8): 2′ O-methyl adenosine
  modified_base (9)..(9): LNA
  modified_base (10)..(11): 2′ O-methyl guanosine
  modified_base (12)..(12): LNA
  modified_base (13)..(13): 2′ O-methyl uridine
  modified_base (14)..(15): LNA
tccagagatggtuga

SEQ ID NO: 9 - RNA, 21 nt, Homo sapiens
uucacaguggcuaaguuccgc